(19) United States
(12) Patent Application Publication
(10) Pub. No.: US 2015/0268208 A1
(43) Pub. Date: Sep. 24, 2015

(54) HYBRID SENSOR ARRAY

(71) Applicant: NANOSENSE INC., Mountain View, CA (US)

(72) Inventors: PAUL A. RHODES, West Palm Beach, FL (US); SAMUEL M. KHAMIS, San Francisco, CA (US)

(21) Appl. No.: 14/667,565

(22) Filed: Mar. 24, 2015

Related U.S. Application Data
(63) Continuation of application No. 12/980,223, filed on Dec. 28, 2010, now abandoned.
(60) Provisional application No. 61/290,859, filed on Dec. 29, 2009.

(51) Int. Cl. G01N 33/00; H01L 29/786; H01L 27/28; H01L 51/00; H01L 51/05

(52) U.S. Cl. CPC G01N 33/0031; H01L 51/0049; H01L 51/0558; H01L 27/283; H01L 29/786; G01N 33/0047

(57) ABSTRACT

The present invention provides devices, methods and systems to selectively detect the binding of a molecular species to a biomolecule. In its olfactory sensing application, the hybrid sensor arrays of the present invention provide a high dimensional signature of odorants present that is also readily reversible, together enabling the identification and localization of a source analyte in the presence of the background odorant landscape inherent in a real-world setting.

### CROSS-REFERENCE TO RELATED APPLICATIONS

This application is a continuation of U.S. patent application Ser. No. 12/980,223, filed Dec. 28, 2010, which application claims priority to US. Provisional Patent Application No. 61/290,859, filed Dec. 29, 2009, the disclosure of which is hereby incorporated by reference in its entirety for all purposes.

### BACKGROUND OF THE INVENTION

Field effect transistors (FETs) fabricated from component semiconducting carbon nanotubes (NTs) have been studied extensively for their potential as sensors. A number of properties of these devices have been identified, and different mechanisms have been proposed to describe their sensing behavior. Devices that incorporate carbon nanotubes have been found to be sensitive to various gases, such as oxygen and ammonia, and these observations have confirmed the notion that such devices can operate as sensitive chemical sensors.

U.S. Pat. No. 7,129,554 to Lieber, discloses electrical devices comprised of nanoscopic wires. The nanoscopic wires can be nanotubes, such as single-walled carbon nanotubes. They can be arranged in crossbar arrays using chemically patterned surfaces for direction, via chemical vapor deposition. Chemical vapor deposition also can be used to form nanotubes in arrays in the presence of directing electric fields, in combination with self-assembled monolayer patterns. The nanowires can be functionalized with biomolecular entities and incorporated into field effect transistors (FETs). The devices are useful for analyte detection and chemical analyses.

U.S. Pat. No. 6,958,216 to Kelley, discloses a class of biological sensing devices that include a substrate comprising carbon nanotubes (CNTs), with chemically attached biological molecules. Changes in the electrical conductivity of the attached biological molecules are exploited to detect their interaction with target species, with DNA activated by means of a molecular electron-transfer reaction between DNA-binding donors and acceptors.

Useful sensors of this type should selectively and reliably respond to an analyte of a wide variety of specific types. For example, it may be desirable to selectively sense a specific amine, while not responding to the presence of other amines in the sample. The carbon nanotubes are hydrophobic, and generally non-selective in reacting with analytes. Examples of covalent chemical attachment of biological molecules to nanotubes, including proteins and DNA, are known in the art, although it has not been convincingly demonstrated that useful detection of specific analytes can be accomplished in this way.

In nature, biological olfactory systems are capable of discriminating greater than 10,000 odorants, have hundreds of distinct receptor types. By analogy, in order to achieve or create a similar artificial olfactometry system, a high dimensional sensor array is desired. To date, no such artificial system has been developed which has such high dimensionality.

Thus, in addition to odor analysis, a versatile detection system is needed having the dimensionality required for a high throughput drug discovery platform, diagnostic applications such as body fluid analysis as well as biomedical applications that achieves many purposes and goals. In addition to an artificial olfactometry system, a device designed having the versatility and ability to interact with membrane proteins, detect ligand-receptor interactions, as well as detecting test substances with biological molecules (e.g., olfactory receptors) is needed. The present invention satisfies these and other needs.

### BRIEF SUMMARY OF THE INVENTION

The present invention provides a hybrid sensor array having high dimensionality fostering the selective detection of many different analyte categories. The system is fundamentally an array of digitally addressable carbon nanotube, nanowire, or graphene FET's, each of which is functionalized, that is made selectively responsive, by one or more interaction-mediating molecular species or hybrid molecular constructs, including but not limited to, single strand DNA or membrane proteins embedded in lipid carrier vehicles such as Nanodiscs. The inventive hybrid sensor arrays can be used in a variety of applications including the detection and identification of explosives, contraband, drug discovery, human outgas, medical applications, toxic industrial chemicals, agricultural organics, fugitive emissions and the like. Advantageously, the described class of hybrid sensor arrays combines the sensitivity of nanotube and other nanoscale transistor devices, the durability and potentially low cost of electronic chips and the high dimensionality required to discriminate amongst a large universe of potential analytes. When functionalized with membrane proteins for example, the inventive arrays of FETs allow interrogation of analyte interaction for use in the pharmaceutical industry such as for drug metabolism studies, pharmacokinetics, and metabolite toxicity analysis.

As such, in one embodiment, the present invention provides a hybrid sensor array, the hybrid sensor array comprising:
a first hybrid sensor, wherein the first hybrid sensor has a first nanotube field effect transistor (NT FET) and a first companion transistor, wherein the first companion transistor addresses the first NT FET by gating a flow of current thereto; and
a second hybrid sensor, wherein the second hybrid sensor has a second NT FET and a second companion transistor, wherein the second companion transistor addresses the second NT FET by gating the flow of current thereto.

In another embodiment, the present invention provides a method for detecting an analyte, comprising:
contacting the analyte with a hybrid sensor array, the hybrid sensor array comprising:
a first hybrid sensor, wherein the first hybrid sensor has a first nanotube field effect transistor (NT FET) and a first companion transistor, wherein the first companion transistor addresses the first NT FET by gating a flow of current thereto;
a second hybrid sensor, wherein the second hybrid sensor has a second NT FET and a second companion transistor, wherein the second companion transistor addresses the second CNT FET by gating the flow of current thereto;
affecting the electric field (e.g. current flow) of the first and the second NT FET to generate a signal; and
detecting the signal thereby detecting the analyte.

In still another embodiment, the present invention provides a distributed leak detection system of a defined geographical location to enable identification of a leak source, the leak detection system comprising:
a plurality of hybrid sensor arrays, wherein each of the hybrid sensor arrays comprises a first hybrid sensor, wherein the first hybrid sensor has a first nanotube field effect transistor (NT FET) and a first companion transistor, wherein the first companion transistor addresses the first NT FET by gating a flow of current thereto;
a second hybrid sensor, wherein the second hybrid sensor has a second NT FET and a second companion transistor, wherein the second companion transistor addresses the second NT FET by gating the flow of current thereto; and
the plurality of hybrid sensor arrays comprising at least one member selected from the group consisting of a stationary, a hand held, a mobile and a portable hybrid sensor array, wherein the plurality of hybrid sensors are recognizable on a map of the defined geographical location, wherein a leak affects the current flow of one of the plurality of hybrid sensors to generate a signal thereby detecting the leak.

These and other aspects, objects and embodiments will become more apparent when read with the detailed description and figures which follow.

### BRIEF DESCRIPTION OF THE DRAWINGS

FIG. 1 A-B illustrate one embodiment of a hybrid sensor array.

FIG. 2 illustrates an array of hybrid sensors comprising a plurality of hybrid sensors, wherein each hybrid has a NT FET and a companion transistor.

FIG. 3 A-B illustrate a schematic of one embodiment of a sensor chip module of the present invention.

FIG. 4 is a schematic of one embodiment of a sensor chip wafer.

FIG. 5 is a schematic of one embodiment of the hybrid sensor array system of the present invention.

FIG. 6 is a schematic of one embodiment of a hybrid sensor array adapted to a specific application of the present invention.

FIG. 7 is a schematic of one embodiment of a hybrid sensor array adapted to a specific application device of the present invention.

FIG. 8 shows a graph of data of source/drain current (▪) across the range of gating voltages immediately after fabrication. The I/Vg curves (▴) were collected following post processing to remove the metallic nanotubes in the transistor channel.

FIG. 9 illustrates an atomic force microscopy images and an inset depicting the molecular structure of a nanodisc.

FIG. 10 illustrates transduction of putative ligand binding to the GPCR rhodopsin by observing an induced change in source-drain current in a carbon nanotube FET.

FIG. 11 illustrates one embodiment of the reversibility of a sensor array of the present invention.

FIG. 12 shows one embodiment of the distributed sensor system of the present invention.

### DETAILED DESCRIPTION OF THE INVENTION

The present invention provides devices, methods and systems to selectively detect analytes in a complex chemical environment. In certain aspects, the hybrid sensor arrays of the present invention provide high dimensionality that is capable of repeated re-sampling, which in turn enables the identification and location of a source analyte all within the complexities and background of a real-world setting. In addition, the present invention provides a signal processing system capable of utilizing signals from these highly dimensional hybrid sensor arrays to rapidly identify odorants within unknown mixtures in the presence of time-varying background. Moreover, in certain aspects, the present invention is portable and mobile and is adapted to communicate to a centralized computing platform and to execute the signal processing system on a multitude of geographically dispersed devices networked to a central computing server.

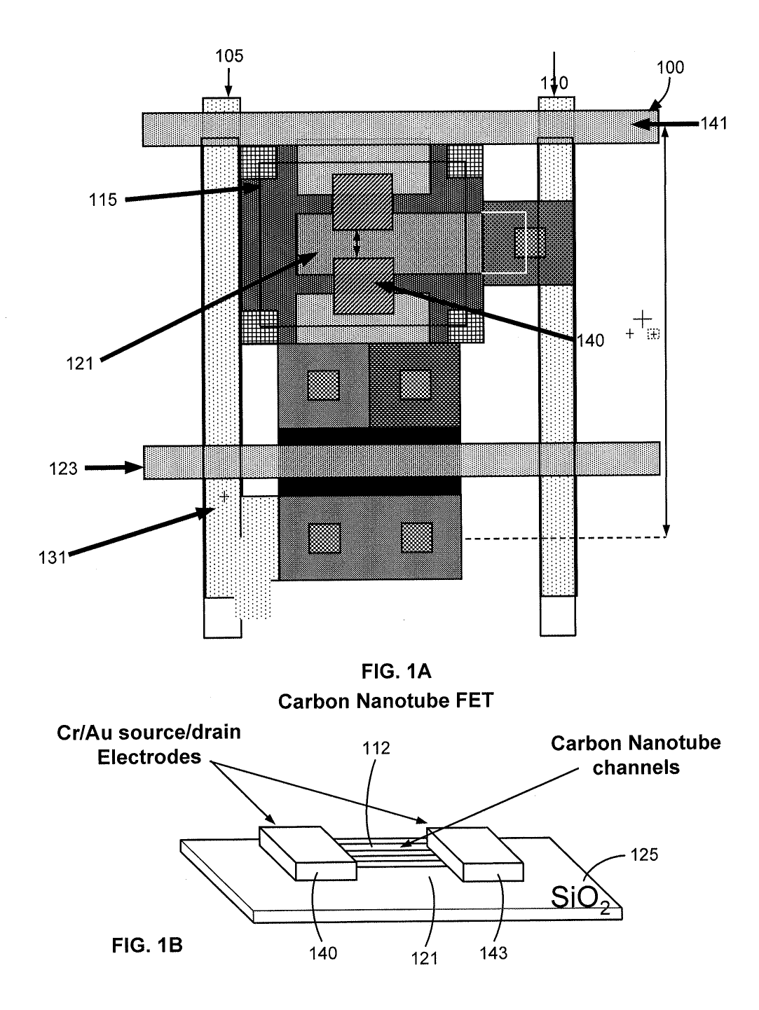

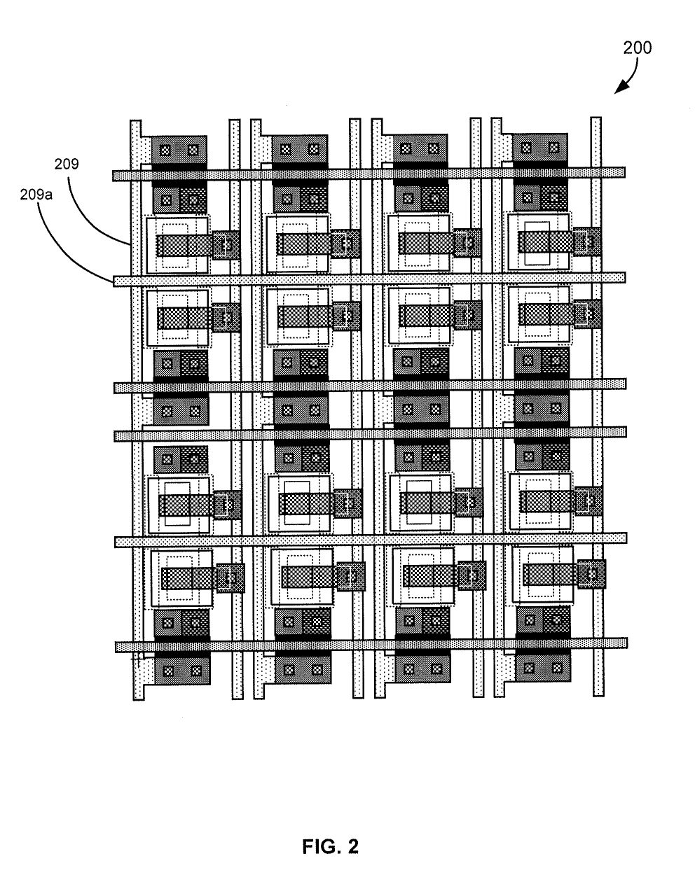

FIG. 1A schematically represents one embodiment of the present hybrid sensor array. The NT FET 115 (shown in 1B) uses for example a carbon nanotube 112 as a transducer. Each nanotube can be functionalized with a front end interaction moiety such as a biomolecule (e.g., a single stranded nucleic acid). The hybrid sensor array is fundamentally a detection and analysis platform that can change its application based upon its functionalization interaction moiety e.g., a biomolecule. FIG. 1B shows an expanded view of a carbon nanotube FET (CN FET), wherein the nanotube 112 is electrically connected to a source electrode 140 and a drain electrode 143 on a gate electrode. In certain aspects, the nanotube 112 is formed of carbon. The source 140 and drain 143 can be made of a metal such as chromium or gold. The nanotube 112 can be formed with a diameter of approximately 5 to 40 nanometers (nm) and with a length of approximately 2-5 micrometers using semiconductor fabrication techniques. The nanotube or sensor element 112 rests on the surface 125 or can be suspended above the surface 125. The nanotube 112 located between the source 140 and the drain 143 forms the channel of a field effect transistor. Optionally, a passivation layer 125 such as SiO₂, may cover the gate electrode 121, which typically comprises silicon or other suitable material. In FIG. 1 A, the NT has bias 141. In addition, NT gate bus 110 allows for opening and closing a plurality of gates within the array.

Each NT FET 115 has a companion transistor 131 that provides an address for the NT FET 115. Each FT 131 within the array receives bias 105 and a gate 123.

In certain embodiments, the present invention provides at least two hybrid sensors i.e., a first hybrid sensor, wherein the first hybrid sensor has a first nanotube field effect transistor (NT FET) and a first companion transistor, wherein the first companion transistor addresses the first NT FET by gating a flow of current thereto; and a second hybrid sensor, wherein the second hybrid sensor has a second NT FET and a second companion transistor, wherein the second companion transistor addresses the second NT FET by gating the flow of current thereto.

In certain aspects, the nanotube of the first and the second NT FET may be the same or different. Suitable nanotubes include, but are not limited to, a single conducting nanotube, a single semiconducting nanotube, a mat of semiconducting nanotubes, and a mat of a combination of an intercalated semiconducting NT and a metallic nanotube. In certain preferred aspects, the first NT FET and the second NT FET are each a carbon nanotube (CNT) FET. The first CNT FET and the second CNT FET may be the same or different. Suitable carbon nanotubes include a single wall carbon nanotube (SWCNT), a multiwall carbon nanotube (MWCNT), graphene and a mixture thereof.

In certain instances, the hybrid sensor array is disposed on a substrate. Suitable substrate materials include, but are not limited to, a dielectric material, an insulating material, a semiconducting material and a conducting material. In certain preferred aspects, disposed on the substrate is each of the NT FETs of the hybrid sensor array comprising: a gate electrode; an insulating layer; a source electrode; and a drain electrode, wherein a nanotube is in contact with the source electrode and the drain electrode. In certain aspects, the substrate is the gate electrode. In operation, a gate voltage is applied to the gate electrode and a bias voltage is applied to the source electrode and the drain electrode and the nanotube conducts current between the source and the drain electrodes.

In certain aspects, the first companion transistor gates current flow to the first NT FET. Similarly, the second companion transistor gates current flow to the second NT FET.

In certain preferred aspects, the first and the second NT FET are each functionalized with a biomolecule. The functionalized biomolecule can be covalently or noncovalently bound to the nanotube. In operation, the functionalized biomolecule interacts with an analyte and interaction affects the electric field (e.g., current flow) of the NT FET such as a CNT FET. As used herein, the term “analyte” includes a chemical compound or odorant molecule, a plurality of chemical compounds, e.g., odorant molecules, which are the subject of an analysis using the sensors herein.

Functionalizing the nanotube comprises attaching at least one biomolecule to exposed nanotube 112. Functionalizing nanotubes is well known in the art (see, WO 2006/110347 published 29 Mar. 2006). Preferred techniques for functionalizing exposed nanotube portion 112 include chemical functionalization. Chemical functionalization includes covalent functionalization or noncovalent functionalization, which modifies and/or adds chemical groups to the surface of exposed nanotube 112. Any chemical reaction known in the art can be used to functionalize exposed nanotube portion 112. An altered current flow is accompanied by interaction with an analyte of interest and the functionalized NT FET.

A wide range of biomolecules are suitable for functionalization. These include, for example, a protein, a peptide, lipid membranes that contain one or more membrane proteins, including for example an olfactory receptor (see, T H Kim et al. Adv. Mater. 2009, 21, 91-94), a ligand, a receptor, a liposome, a micelle, a drug delivery vehicle, an enzyme substrate, a hormone, an antibody, an antigen, an hapten, a carbohydrate, an oligosaccharide, a polysaccharide, a nanodisc, a nucleic acid, an oligonucleotide, a deoxyribose nucleic acid, a fragment of DNA, ribose nucleic acid, a fragment of RNA, siRNA, miRNA, nucleotide triphosphates, and a PNA. A preferred biomolecule is a nucleic acid oligonucleotide, such as DNA. Table 1 contains a list of suitable oligonucleotides for use in the present invention.

TABLE 1

| | | |
|---|---|---|
| Seq1 | GAGTCTGTGGAGGAGGTAGTC | SEQ ID NO: 1 |
| Seq1 α | AAAACCGGGGGGGGGGTTTTT | SEQ ID NO: 2 |
| Seq 1 RNA | GAGUCUGUGGAGGAGGUAGUC | SEQ ID NO: 3 |
| Seq 1R1 | CGAGGGAGTTGTACTTGGAGG | SEQ ID NO: 4 |
| Seq 1R2 | TGATGTGGGTGCCGAAGGTGA | SEQ ID NO: 5 |
| Seq 2 | CTTCTGTCTTGATGTTTGTCAAAC | SEQ ID NO: 6 |
| Seq 2 α | AAAACCCCCGGGGTTTTTTTTTTT | SEQ ID NO: 7 |
| Seq 2 Soup | 4dATP: 5dCTP: 4dGTP: | — |
| | 11dTTP | |
| Seq2 RNA | CUUCUGUCUUGAUGUUUGUCAAAC | SEQ ID NO: 8 |
| Seq 2R1 | TACTGTCTCATTCTGGATATTCTG | SEQ ID NO: 9 |
| Seq 2R2 | GAATATGTACTTGTCCCTGTTCTT | SEQ ID NO: 10 |
| GT12 | GTGTGTGTGTGTGTGTGTGTGTGT | SEQ ID NO: 11 |
| A21 | AAAAAAAAAAAAAAAAAAAAA | SEQ ID NO: 12 |
| C21 | CCCCCCCCCCCCCCCCCCCCC | SEQ ID NO: 13 |
| G21 | GGGGGGGGGGGGGGGGGGGGG | SEQ ID NO: 14 |
| T21 | TTTTTTTTTTTTTTTTTTTTT | SEQ ID NO: 15 |
| Seq1 | GAGTCTGTGGAGGAGGTAGTC | SEQ ID NO: 1 |

In a preferred aspect, a useful DNA oligonucleotide is an 18 mer (18 nucleotides long) to about a 30 mer, such as 18, 19, 20, 21, 22, 23, 24, 25, 26, 27, 28, 29, 30 mer. Preferably, the oligo has a known sequence of about 21 nucleotides, which is specific for an analyte of interest. In certain preferred aspects, the oligonucleotide is a single stranded DNA (ssDNA). ssDNA provides enormous dimensionality as analyte binding is sequence-specific, i.e., specific analytes bind specifically to known sequences. Further, binding is low-affinity and so reverses in seconds or less with brief intake. Moreover, the devices, systems and methods herein allow repeated resampling and ssDNA is durable.

In operation, the binding of an analyte to the oligo causes an electric charge to pass from the biomolecule to carbon nanotube 112. Carbon nanotube 112 conducts the electric charge to FET 115. The hybrid sensor preferably transmits such an electric charge to a detection module such as a device suitable for recording and analyzing the charge. An altered current flow in the first and second NT FET is detected by a detection module. Analyzing the altered current flow can be accomplished for a wide variety of applications.

In one embodiment, the nanotube 112 is functionalized with a nanodisc. In general, a nanodisc is a nanostructure created using a process of self-assembly of a phospholipid bilayer disc surrounded by a membrane scaffold protein (MSP) belt. Both the size and the chemical nature of the nanodisc can be modified. Nanodiscs furnish a stable artificial functional environment for membrane proteins. They can be attached to the carbon nanotubes comprising the FET channel, and binding or other interactions of analyte molecules with the membrane protein may be detected via the perturbation of transistor properties (for example, current), which allows the sensor arrays herein to be utilized for the detection and measurement of binding or other interaction of test substances with membrane proteins. There are a wide range of applications for such technology, including artificial olfaction (where the ligand is a volatile species and the protein an olfactory receptor) as well as including therapeutics, diagnostics, imaging as well as drug discovery.

Nanodiscs are generally described in U.S. Patent Publication No. 2006/0211093 to Sligar et al. Membrane scaffold proteins (MSP) assemble with target membrane or other hydrophobic or partially hydrophobic proteins to form soluble nanoscale lipid discs within which membrane proteins may be embedded in a setting which preserves their native structure and function. In the presence of phospholipid, MSPs form nanoscopic phospholipid bilayer disks, with the MSP stabilizing the particle at the perimeter of the bilayer domain. The particle bilayer structure allows manipulation of incorporated proteins in solution or on solid supports. This stable native environment for membrane proteins, when coupled to the carbon nanotube FET array described herein, presents a novel electronic binding assay to facilitate pharmaceutical and biological research, structure/function correlation, structure determination, and bioseparation.

In one nanodisc embodiment, a target membrane protein (e.g., G protein-coupled receptor) is included in the membrane scaffold protein/phospholipid mixture. The nanodisc will self-assemble, incorporating the target membrane protein in a configuration wherein its natural structural and functional characteristics are preserved. These nanodiscs can be from about 5 to about 500 nm, about 5 to about 100 nm or about 5 to about 50 nm in diameter. These structures comprising phospholipid and membrane scaffold proteins preserve the overall bilayer structure of normal membranes, but provide a system which is both soluble in solution and which can be assembled or affixed to a variety of surfaces.

Turning now to FIG. 2, illustrated is an array of hybrid sensors 200 comprising a plurality of hybrid sensors, wherein each hybrid sensor has a NT FET 209 and a companion thin film TFT 209a. Array of hybrid sensors 200 shows 16 CNT FETs and 16 companion TFTs. A skilled person will understand that although FIG. 2 shows an array of 16 hybrid sensors, this is but one embodiment. In certain instances, the hybrid sensor array consists of at least n hybrid sensors, wherein the value of n is a number in the range of about 2 to about 10⁵, or more. In other instances, the value of n is between at least 8 hybrid sensors to at least 64 hybrid sensors. In other instances, the values of n is at least 16 hybrid sensors. In certain preferred instances, the at least 16 hybrid sensor array is a first hybrid sensor array in a first well of a multiwell sensor module, wherein each sensor of the at least 16 hybrid sensor array of the first hybrid sensor array is functionalized the same (e.g., same biomolecule). In another aspect, the sensor module has a second at least 16 hybrid sensor array in a second well of the multiwell sensor module, wherein each sensor of the hybrid sensors of the second hybrid sensor array has a different functionalization than the first at least 16 hybrid sensor array (e.g., different biomolecule). A skilled artisan will appreciate that the multiwell sensor module has y multiwells, wherein the value of y of the multiwell sensor module is for example 2 wells, 8 wells, 32 wells, 64 wells, 96 wells, 384 wells, in certain instances, the values of y can be between about 2 to about 10⁵. In a preferred aspect, the hybrid sensor array 200 has 16 hybrid sensors for each well of a sensor chip module.

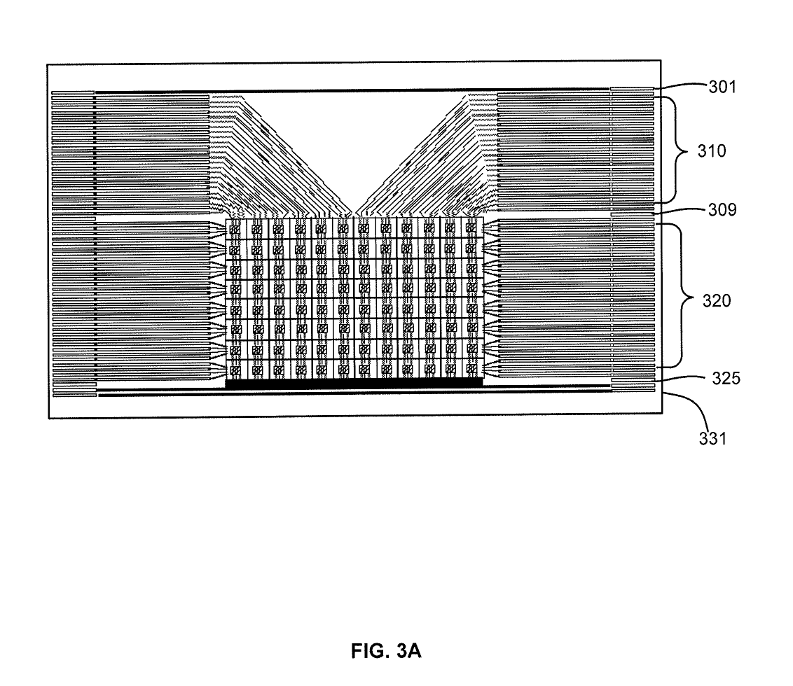

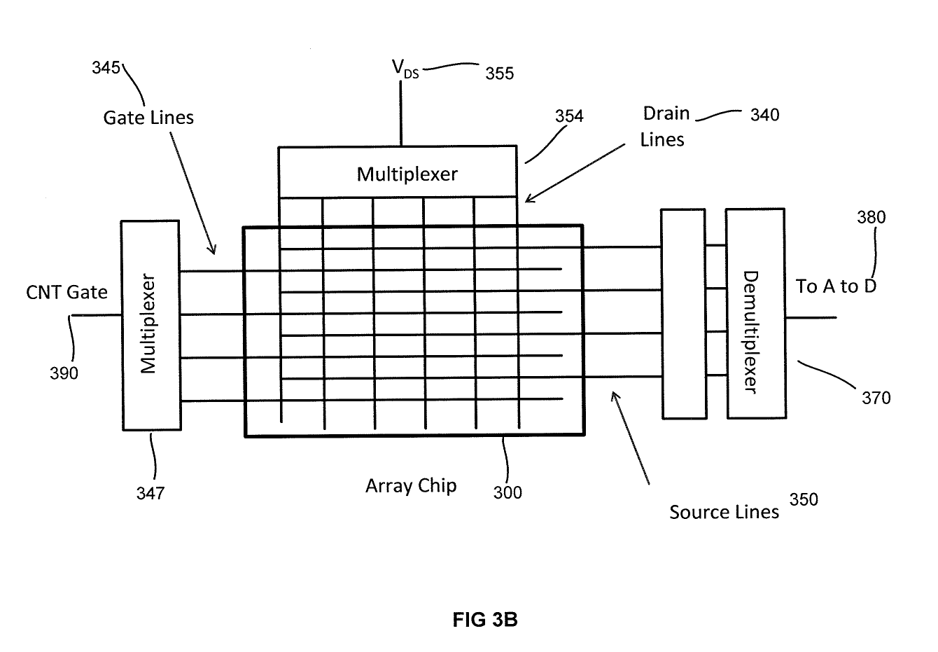

FIG. 3A is a schematic of one embodiment of a sensor chip module 300 of the present invention. In this specific embodiment, sensor chip module 300 has 96 wells (8×12 matrix) 305, wherein each well comprises an array of 16 hybrid sensors (1536 hybrid sensors). Each of the 16 CNT FET in the array of 1 well of the sensor chip module 300 has a distinct biomolecule (e.g. a single stranded nucleic acid (ssDNA)) functionalized on a CNT. Sensor chip module 300 can be for example, 17.5 mm by 28.5 in dimension. Those of skill in the art will know of other dimensions suitable for use in the present invention. In one embodiment, sensor chip module has TFT ground 301, TFT gate tabs 310, ground 309, TFT data tabs 320 ground 325 and common bias 331.

In certain aspects, in a hybrid sensor array of the present invention, the first companion transistor gates current flow to (e.g., on/off) the first NT FET. Similarly, the second companion transistor gates current flow to (e.g., on/off) the second NT FET. During operation, when the NT FET detects an analyte there is a concomitant altered current flow in the first and second NT FET of the sensor module. This altered current or signal is detected by a detection module. The altered current flow is accompanied by interaction with an analyte and the functionalization of the NT FET.

In certain embodiments, the systems of the present invention comprise a sensor chip module e.g., 300, a detection module and a data processing module. One embodiment of the detection module is shown in FIG. 3B. As shown therein, TFT gate voltages as well as CNT FET source 350 drain signals 340 and gate signals 345 are applied to the chip by digital to analog converters (DAC's) using control electronics. In certain aspects, the signals are typically between −10 to 10 volts for the CNT source drain or gate signal and between −10 and +20 volts for the TFT gates. These signals are applied to the chip through flexible printed circuit tabs. The signals leaving the chip also leave on flexible printed circuit tabs, and are immediately passed through transimpedance amplifiers (e.g., a current to voltage converter), which amplifiers supply a virtual ground to the devices so that they are not being abruptly switched from ground to voltage. These voltages are then passed into a multiplexer and subsequently to an analog to digital converter 380.

In one embodiment, the array is designed without a companion thin film transistor for each CNT FET. FIG. 3B shows a sensor array addressed by two banks of multiplexers 347 and 354 and one demultiplexer 370. The source drain voltage for the array is applied at the input of one of the multiplexers 355, which is then used to address a single column of the array being biased at a given time. At the same time, all the rows of the array leave the chip and go through trans impedance amps supplying a virtual ground for every device. The feedback voltage of the trans impedance amps are demultiplexed 370 and that output is then sent to an analog to digital (A/D) converter 380. The second multiplexer 347 controls the gate voltages for the CNT FET's on the chip. A CNT gate voltage 390 will be applied to the input of a multiplexer connected to the lines running parallel to the source outputs on the rows of the array. This multiplexer addresses which row is gated and with a given voltage. Only the devices that have been activated by the other two addressing lines are effectively gated.

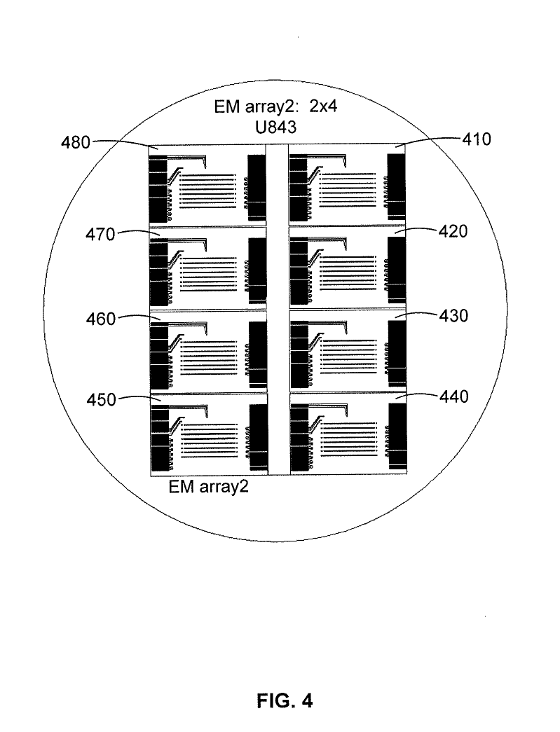

Turning now to FIG. 4, which is a schematic of one embodiment of a sensor chip wafer 400 having 8 hybrid sensor array modules, each module having 96 hybrid sensor arrays. Each module, 410, 420, 430, 440, 450, 460, 470, and 480 has 96 hybrid sensors on board. In this preferred embodiment, each NT in the 96 hybrid sensor array has the same functionalization. In this embodiment, the 96 hybrid sensor array in 410 has a ssDNA oligo functionalized to the CNT of the FET. However, the ssDNA of 410 is different than the ssDNA of 420 and so on. In this embodiment, there are 8 different ssDNA molecules on these sensors.

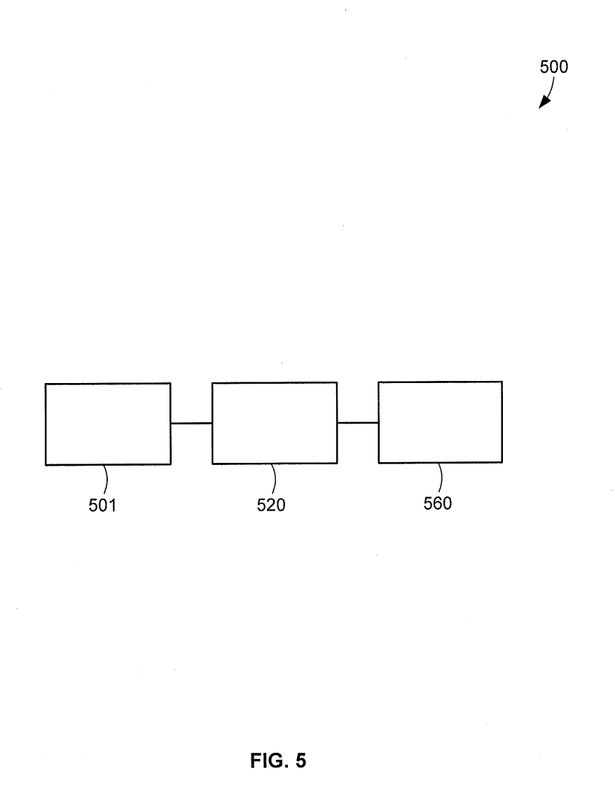

FIG. 5 is a schematic of one embodiment of the hybrid sensor array system 500 of the present invention. As shown therein, the sensor module 501 comprises the hybrid sensor array such as a multiwell module wherein each well contains about 16 hybrid sensors. In addition to the sensor module, the hybrid sensor array system further comprises a detection module 520 and a data processing module 560. The detection module 520 is preferably a device suitable for recording and analyzing the altered current flow.

The data processing module 560 provides the capabilities to enable the programmed operation of the sensors and to receive, store, and provide useful analysis and display of the data that is obtained. Typically, such processing capability is at least partially provided through an external computer that is electrically coupled (via electrical cable or other operational connection) to the power supply and monitoring portion of the system. An operational connection can include one or more wires or cables, an infrared transmitter and receiver, a radio transmitter and receiver or any other connection typically used to transmit information between a central processor and a peripheral device or appliance. In addition to that portion of the processing capability provided by the computer, additional processor(s) can be integrated into the hybrid sensor array system itself. By way of example, instruction sets that are routinely run during set up, operation and shut down are conveniently integrated into a hybrid sensor array system. These typically include power adjustments, diagnostic operations, and the like.

Typically, the computer includes appropriate programming to automate operation of hybrid sensor array system, as well as its ancillary components, e.g., including instructing the power supply in applying, regulating and/or modulating the current applied to the hybrid sensor array. The computer also will include programming that enables the receipt and storage of conductance data received from the hybrid sensor array system in response to the application of sample analyte with the sensing surface. In addition, the computer will also typically be programmed to provide meaningful analysis of the conductance data.

The hybrid sensor arrays of the present invention are useful in a wide variety of applications. The applications include, but are not limited to, bomb detection, environmental toxicology and remediation, biomedicine, cosmetic and/or perfume, pharmaceutical, transportation, emergency response and law enforcement, detection, identification, and/or monitoring of combustible gas, emissions control, air intake, smoke, hazardous leak, hazardous spill, fugitive emission, beverage, food, and agricultural products monitoring and control, such as freshness detection, fermentation process, and flavor composition and identification, detection and identification of illegal substance, explosives, diesel/gasoline/aviation fuel, body fluids analysis, drug discovery, infectious disease detection and breath applications, worker protection, arson investigation, personal identification, perimeter monitoring, fragrance formulation, and solvent recovery effectiveness, shipping container inspection, enclosed space surveying, product quality testing, materials quality control, product identification and quality testing.

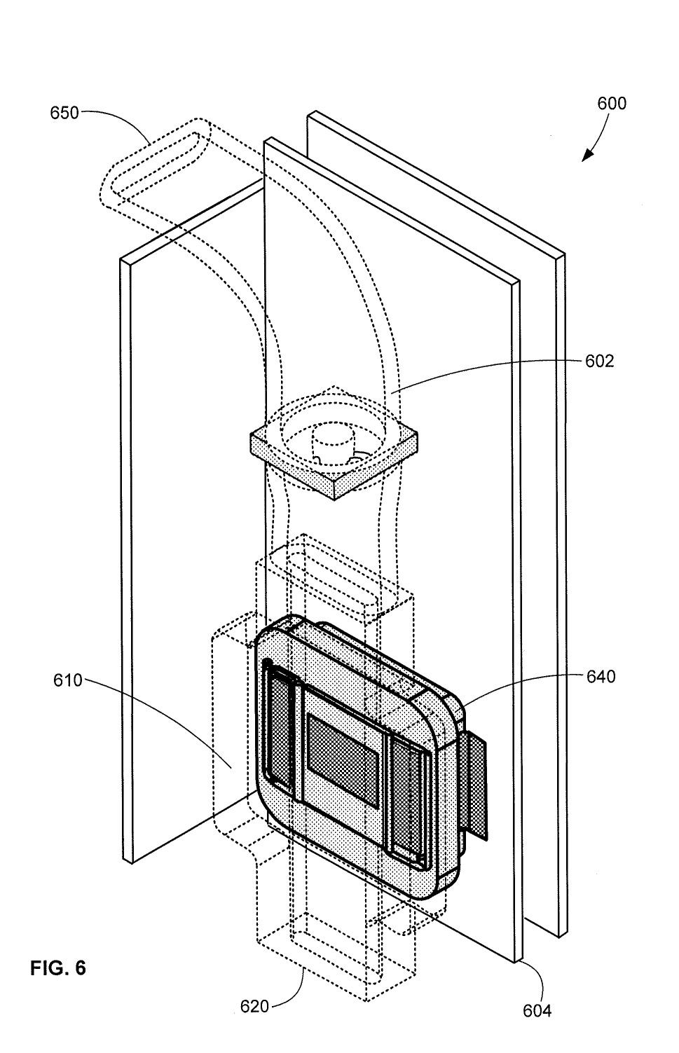

FIG. 6 is a schematic of one embodiment of a hybrid sensor array adapted to a specific application device 600 of the present invention. In this adaptation, hybrid sensor array module is enclosed in compartment 640. Intake 620 allows air or other gas to flow into headspace 610 and across sensor compartment 640. Air or gas then moves through space 602 and outlet 650. The device 600 is mounted on surface 604. The device is useful for detection, identification, and/or monitoring of combustible gas, emissions control, air intake, smoke, hazardous leak, hazardous spill, and agricultural products monitoring and control.

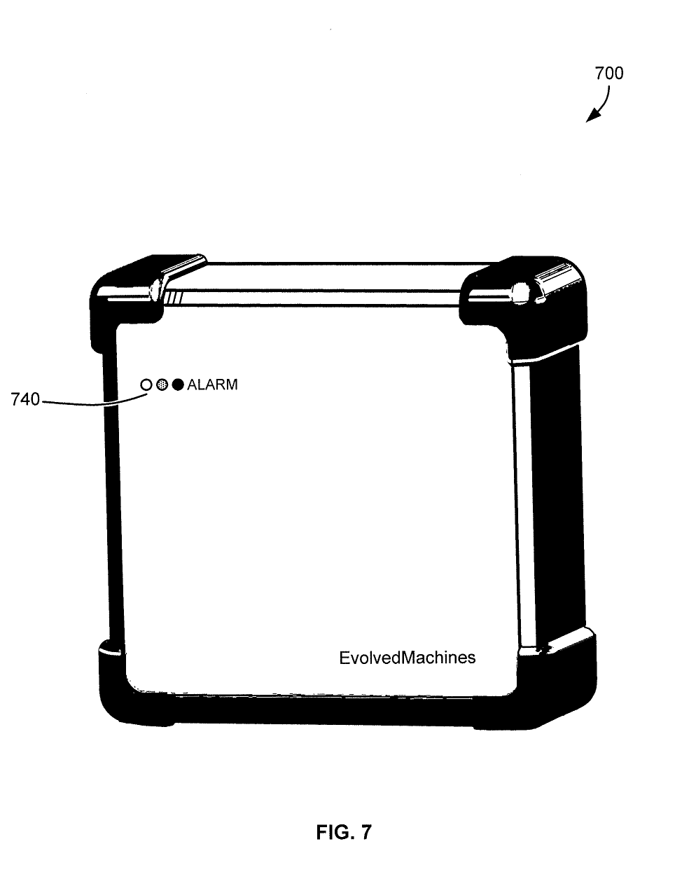

FIG. 7 is a schematic of one embodiment of a hybrid sensor array adapted to a specific application device 700 of the present invention. The device is portable and contains a hybrid sensor array within the enclosed box. The device 700 is ideal for shipping container inspection as in operation it is affixed to a shipping container to detect hazardous substances and illicit contraband. Alarm 740 signals when a hazardous substance is detected.

In another embodiment, the present invention provides a distributed leak detection system for a defined geographical location to enable identification of a leak source. Example 4 illustrates one embodiment of a distributed autonomous leak detection system of the present invention. In certain embodiments, the leak detection system of the present invention comprises: a plurality of hybrid sensor arrays as described herein, wherein the plurality of hybrid sensor arrays comprising at least one member selected from the group consisting of a stationary, a hand held, a mobile and a portable hybrid sensor array, wherein the plurality of hybrid sensors are recognizable on a map of the defined geographical location, wherein a leak affects the current flow of one of the plurality of hybrid sensors to generate a signal thereby detecting the leak.

Preferably, the distributed leak detection system comprises a network, wherein each of the plurality of hybrid sensor arrays comprises a network interface to communicate to the network. For example, the network can be the Internet. In addition, the sensor arrays can be configured to communicate to the network via a direct wire link (e.g., USB cable, firewire cable), a direct wireless link (e.g., infrared, Bluetooth), or a wired or wireless network link such as a local area network (“LAN”), a wide area network (“WAN”), a wireless wide area network (“WWAN”), or an Institute of Electrical and Electronics Engineers 802 (“IEEE 802”) wireless network such as an IEEE 802.11 (“WiFi”) network. The distributed leak detection system of the present invention comprises a monitoring center wherein the monitoring center comprises a data processing module which aggregates data from the plurality of hybrid sensor arrays and can be displayed.

In certain embodiments, each hybrid sensor (or other sensor) has a communication address recognizable by the computer network. In certain preferred embodiments, the monitoring center communicates with each sensor in the network via the communication interface using either wireless or wired technologies. Wireless technologies can include infrared, radio waves, satellite and microwaves. In certain aspects, the sensor arrays have wireless ethernet capabilities such as a radio card having a media access controller. In certain aspects, the media access controller regulates the data from the sensor array to the network.

In other embodiments, the network include hardwired communication circuits where the sensor is physically connected by wires to other communications devices or communication systems such as telephone lines, satellite or wireless communication devices, and the like. Additionally or alternatively, the communication unit of an individual sensor can include short-range wireless capabilities for communication with local alert and/or long-range communication devices such as telephones, private or public networks, cellular communication networks or satellite devices that may preexist or be installed for communication with a sensor or a plurality of sensors such as hybrid sensors. Such short-range wireless devices include communication devices utilizing unregulated spectrums using existing protocols such as Bluetooth. Alternatively, wireless LAN protocols such as dictated by IEEE Standard 802.11(b) or 802.11(g) are also used.

A network of sensors as described herein can be configured to communicate with a plant monitoring system, e.g., a server, and/or sensor unit communicate with each other as a distributed network, using communication components known in the art. In this way, for example, a first sensor generates preliminary data if it detects a leak or possibly a larger event and can trigger a neighboring second sensor via the distributed network to make a confirmation measurement. The communication can include on-site alerts such as optical (indicator lights), audible alerts (e.g., alarm sounds), tactile (e.g., vibration of the unit) or can be interfaced to an appropriate control valve for simply shutting off the supply upon the detection of emergency events, for instance.

In certain preferred aspects, a wireless module is connected to each hybrid sensor array (or other sensor) in order to transmit data wirelessly from the sensor to the network. Sensor data can be transmitted from the wireless module to a receiver. The sensor array optionally comprises a Global Positioning System (GPS) device. The term Global Positioning System (GPS) generally refers to the worldwide radio-navigation system that uses the position of satellites to determine locations on the earth. The GPS is formed generally from a group or constellation of orbiting man-made satellites and their respective ground station, thereby utilizing such satellites as reference points to calculate accurate positions.

The distributed sensor system has a plurality of hybrid sensor arrays. Other sensors suitable to network include those sensors known in the art such as conducting polymer films, semiconducting polymer sensors, surface acoustic wave devices, fiber optic micromirrors, quartz crystal microbalances, optical fibers and combinations thereof.

The monitoring center comprises data processing capabilities to enable the programmed operation of the sensors within the network and to receive, store, and provide useful analysis, aggregation of data and display of the data that is obtained. Typically, such processing capability is at least partially provided through an external computer that is electrically coupled (via electrical cable or other operational connection) to the power supply and monitoring portion of the distributed system. An operational connection can include one or more wires or cables, an infrared transmitter and receiver, a radio transmitter and receiver or any other connection typically used to transmit information between a central processor and a peripheral device or appliance.

In a preferred aspect, the plurality of hybrid sensors are recognizable on a map of the defined geographical location. To provide the location for a leak, the location of reporting sensors are known. In a fixed system, the location of each sensor can be surveyed at the time the sensors are placed. In a system with moving or re-locatable sensors, each sensor typically self-surveys with a global positioning system receiver (“GPS”) or other such system. The sensor array information associated with each of the plurality of sensors of the distributed system are overlaid on a geographical map displayed on a graphical computer interface (e.g., a web browser) of a user.

### Examples

Example 1 illustrates certain electronic characteristics of one nanotube mat FET on a nanotube FET sensor chip.

A hybrid sensor array was constructed containing 16 nanotube FETs along with a companion TFT in a 96-well sensor module and the attendant electronics to control gate voltage wherein each transistor is individually controllable and the source/drain voltage (common across the chip) and to read the current from each of the 1,536 sensor transistors. The 16 transistors in each well is identically functionalized, allowing the S/N benefit of averaging, while the 96 wells on each chip affords high dimensionality in this artificial “olfactory epithelium”. A single breadboard airpath contains 3 such chips in series, along with the attendant control electronics.

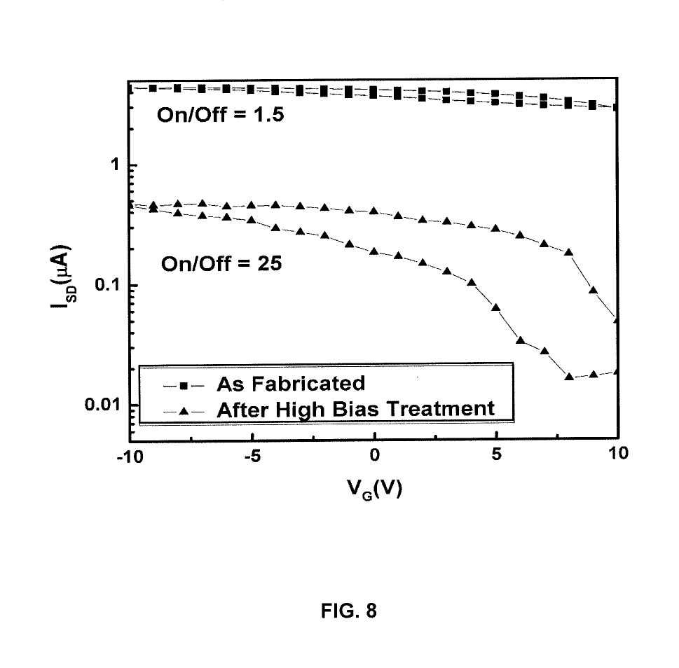

In FIG. 8, the data show the source/drain current (▪) across the range of gating voltages immediately after fabrication. The high current level and low on-to-off current ratio is indicative of a high metallic content in the nanotubes before postprocessing. The I/Vg curves (▴) were collected following postprocessing to remove (burn out) the metallic (i.e. non-semiconducting nanotubes) present in the transistor channel. The resulting lower source/drain current, and higher on/off current ratio (here 25) indicates a functioning nanotube mat transistor (“mat” signifies that each channel consists of many nanotubes) ready for functionalization with a biomolecule.

Example 2 illustrates methods and procedure to functionalize nanotube mats with membrane fragments and Nanodiscs.

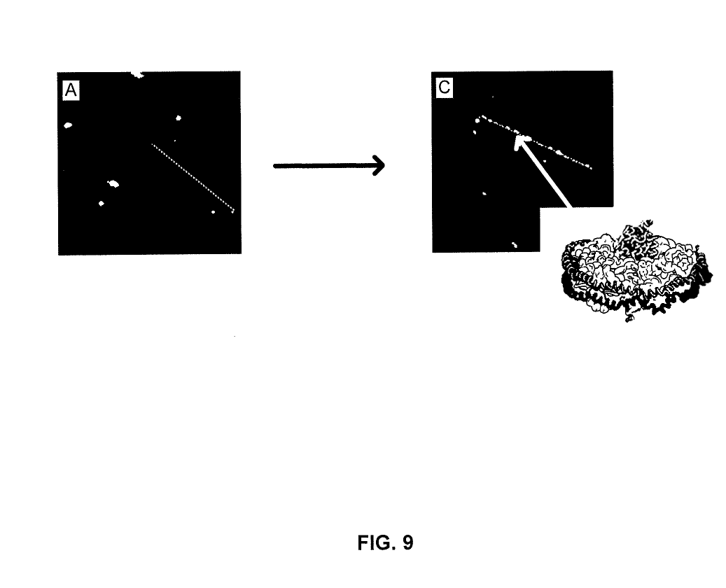

A method was developed to induce the selective attachment of Nanodiscs, which are shown as white spots in the atomic force microscopy images here on FIG. 9. FIG. 9 further shows an inset depicting the molecular structure of a Nanodisc, a 10 nm diameter self-assembling lipid bilayer confined by a self-assembling protein collar, here shown incorporating a membrane protein.

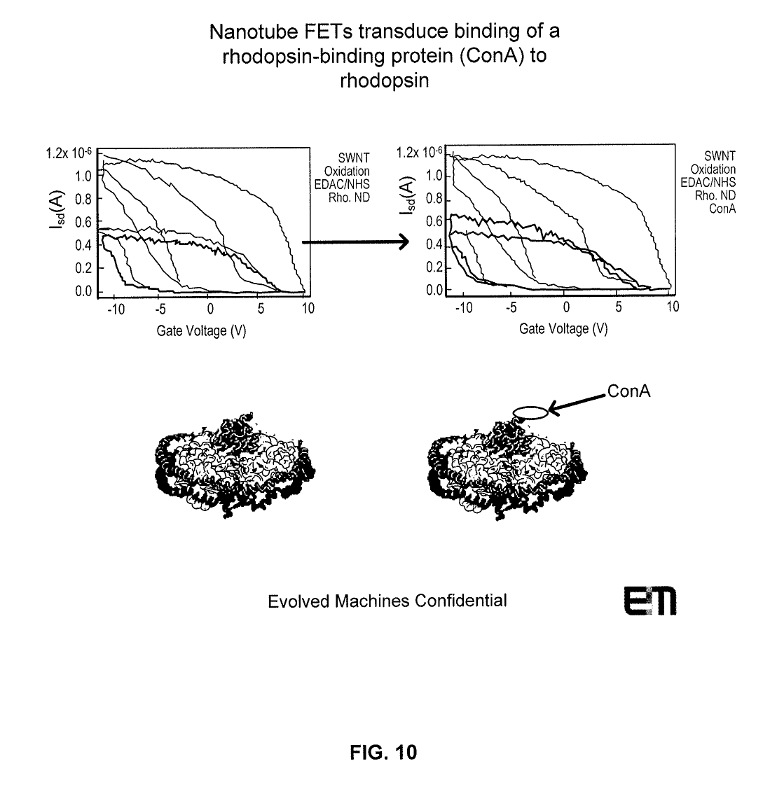

FIG. 10 illustrates transduction of putative ligand binding to the GPCR rhodopsin by observing an induced change in source-drain current in a carbon nanotube FET. The thick trace depicts a typical source-drain voltage profile for a nanotube FET with rhodopsin-containing Nanodiscs attached as gate voltage is swept from off to on and back. The lighter traces depict the so-called I/Vg curve at earlier stages in the process, where the nanotubes are bare and where the chemical steps taken to induce Nanodisc attachment are performed (intermediate curves). In FIG. 10, the thick trace in the graph on the right depicts the shift in voltage (approximately 20%) resulting when the nanotube FET is exposed to the rhodopsin ligand ConA, demonstrating an electronic readout of binding to a membrane protein.

Example 3 illustrates detection and identification of certain analytes. Using a hybrid sensor of the present invention, various analytes were detected. The head pressure of each analyte a 3% concentration was detected with the ssDNA molecules of Table 1. The table shows the values of change in gate current.

TABLE 2

| Performed at 3% | | | | | | | | |
|---|---|---|---|---|---|---|---|---|
| of saturated | | | | | | | | |
| vapor pressure | Bare | Seq1 | Seq1 α | Seq1 RNA | Seq1R1 | Seq2 | Seq2α | Seq 2 Soup |
| Propionic Acid | 0 ± 1 | +17 ± 2 | 0 ± 1 | +45 ± 10 | | +8 ± 1 | +10 ± 3 | 0 ± 1 |
| Hexanoic Acid | | 0 ± 4 * | | | | | | |
| Octanoic Acid | | 0 ± 4 * | | | | | | |
| 2-Methyl Valeric | 3 ± 2 | 35 ± 7 | | | | 5 ± 3 | | |
| Acid | | | | | | | | |
| 4-Methyl Valeric | 0 ± 3 | 30 ± 5 | | | | 12 ± 4 | | |
| Acid | | | | | | | | |
| n-Octanal | | −50 ± 5 | | | | | | |
| n-Nonanal | | −13 ± 5 | | | | | | |
| n-Decanal | | 0 ± 4 * | | | | | | |
| DMMP | 0 ± 1 | −14 ± 2 | 0 ± 1 | + 30 ± 5 | | −7 ± 2 | −15 ± 2 | 0 ± 1 |
| DNT | 0 ± 1 | −14 ± 4 | 0 ± 1 | −20 ± 5 | | −4 ± 2 | 0 ± 4 | 0 ± 1 |
| (+) Limonene | 5 ± 2 | 13 ± 4 | | | 50 ± 7 | 40 ± 8 | | |
| (−) Limonene | −7 ± 3 | −7 ± 4 | | | −30 ± 5 | 6 ± 3 | | |
| L-Carvone | −7 ± 4 | −20 ± 5 | | | | −10 ± 4 | | |
| D-Carvone | −8 ± 4 | −10 ± 5 | | | | −15 ± 5 | | |
| Methanol | 0 ± 1 | −12 ± 2 | −50 ± 2 | 32 ± 5 | | −20 ± 2 | 8 ± 1 | −60 ± 4 |
| Trimethylamine | −9 ± 2 | −20 ± 2 | −20 ± 3 | −40 ± 10 | | −30 ± 2 | 14 ± 2 | −25 ± 5 |
| Dimethyl sulfone | | 50 ± 10 | | | | | | |

| Performed at 3% | | | | | | | | |
|---|---|---|---|---|---|---|---|---|
| of saturated | | | | | | | | |
| vapor pressure | Seq2R1 | Seq2R2 | Seq2 RNA | GT₁₂ | A₂₁ | C₂₁ | G₂₁ | T₂₁ |
| Propionic Acid | | | +40 ± 5 | 0 ± 1 | 0 ± 1 | 0 ± 1 | 0 ± 1 | 0 ± 1 |
| Hexanoic Acid | | | | | | | | |
| Octanoic Acid | | | | | | | | |
| 2-Methyl Valeric | | | | | | | | |
| Acid | | | | | | | | |
| 4-Methyl Valeric | | | | | | | | |
| Acid | | | | | | | | |
| n-Octanal | | | | | | | | |
| n-Nonanal | | | | | | | | |
| n-Decanal | | | | | | | | |
| DMMP | | | −35 ±10 | 0 ± 1 | 0 ± 1 | 0 ± 1 | 0 ± 1 | 0 ± 1 |
| DNT | | | −70 ± 10 | 0 ± 1 | 0 ± 1 | 0 ± 1 | 0 ± 1 | 0 ± 1 |
| (+) Limonene | 80 ± 8 | 60 ± 8 | | | | | | |
| (−) Limonene | −65 ± 9 | −80 ± 10 | | | | | | |
| L-Carvone | | | | | | | | |
| D-Carvone | | | | | | | | |
| Methanol | | | 20 ± 5 | −4 ± 2 | −15 ± 2 | 0 ± 1 | −50 ± 2 | 0 ± 1 |
| Trimethylamine | | | −45 ± 5 | −50 ± 5 | −25 3 | −16 ± 5 | −50 ± 6 | −10 ± 6 |
| Dimethyl sulfone | | | | | | | | |

* Detectable at higher Concentration

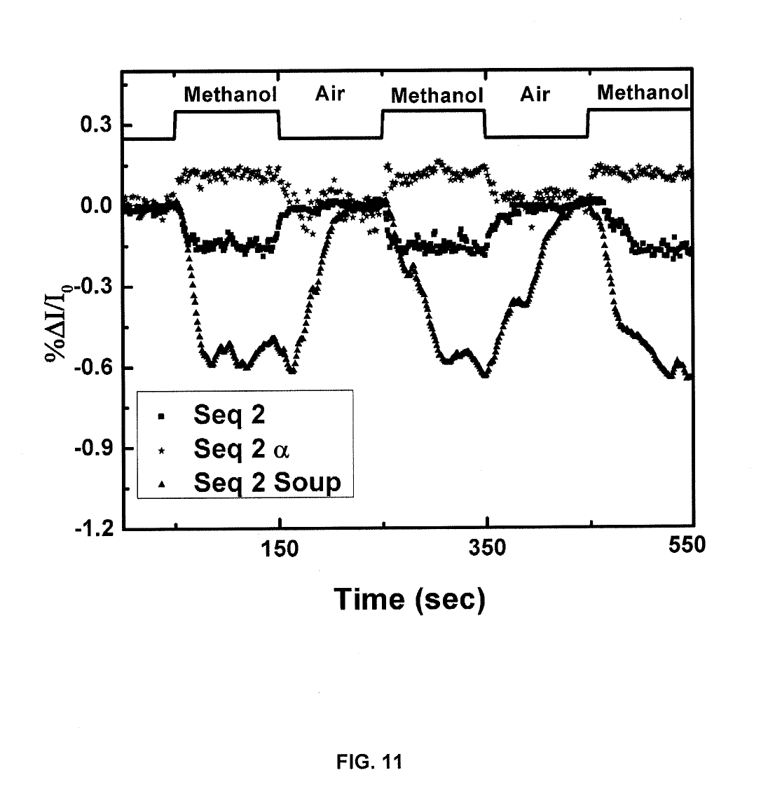

The results of the identification of methanol for three of the sensors is shown in FIG. 11. The reversibility of the sensor array is illustrated in FIG. 11, where sensor response returns to neutral after a few seconds once the analyte (methanol in this example) is removed. The figure also demonstrates that reorganizing (here alphabetizing or randomizing) ssDNA sequence changes both sign and magnitude of sensing response. This is the key enabler for a multi-dimensional sensor array, where each site generates an uncorrelated response, yielding a unique signature for each analyte.

Example 4 illustrates a distributed autonomous leak detection system

This example introduces a chemical sensor system, capable of dynamically identifying diverse target odorants in a complex environment characterized by unknown and changing backgrounds. It is composed of a distributed system of fixed (e.g., wall mounted), hand held, and mobile sensors, with data fused in a dynamic map overlaid on a plant floor map to enable identification of a leak source, to map the course and direction of a leak plume, to thereby guide plant safety reaction measures in real time. The product functional elements are described in the following sections.

### 1. Sensor Array

A central element of the system is a chemical sensor semiconductor chip comprising an array of nanotube-FET's functionalized with a coating of single-strand DNA. It combines the following characteristics, important for a leak detection system capable of addressing many analytes and operating in unknown and a novel background.

### 2. High Dimensionality

The sensor systems capable of detecting a wide range of odorants is highly dimensional. Biological olfactory systems, capable of discriminating more then 10,000 odorants, have hundreds of distinct receptor types. In one embodiment, the devices employ sensor arrays representing more than 100 independent components. These novel sensor arrays have high dimensionality and thus are capable of selective detection of many different analyte categories spanning the target areas of explosives, toxic industrial chemicals, agricultural organics, and more.

### 3. Distributed Representation

Any given odorant triggers a distributed pattern across a high dimensional array, enabling representation of a limitless number of distinct odorants and sensor activity pattern corresponds to molecular motif structure. As in biological olfaction, similar molecules produce correspondingly similar patterns.

### 4. Ability to Identify Analyte Across a 1000-Fold Range of Concentrations

Even indoors, odorant plumes are turbulent and dynamically changing concentration distributions, and so it is vital that a sensor be capable of invariantly identifying analyte identity across a over 1000-fold range of analyte concentrations. In biological olfaction, which is confronted with the same problems, a low concentration of a given odorant triggers a core pattern across the receptor array. As concentration increases, additional receptors are recruited to activity while leaving the core sensor pattern intact. Thus a given analyte is represented not by a single template, but rather a family of odorant patterns which span the relevant range of concentrations. The inventive sensor array likewise respond in an affinity-based fashion and produce distributed patterns that change with increasing concentration in a manner reminiscent of biological sensors.

### 5. Sensor Technology

In one aspect, the sensor is based on a single-walled carbon nanotube field effect transistor as an electronic read-out component and coated with single stranded DNA as the chemical recognition site. Responses of the sensor differ in sign and magnitude for different gases and can be tuned by choosing the base sequence of ssDNA. By changing the ssDNA base sequence, it is possible to obtain an array of sensors, each responding differently to a given analyte. Given low cost and nano-scale size of the devices and the ability to customize each transistor response by a unique ssDNA sequence, it is thus possible to build a very high dimensionality sensor, i.e. a sensor that provides a signature uniquely identifying each different analyte.

### 6. Reversibility

The inventive sensors have rapid response and recovery times on the scale of seconds, which allows detecting very small analyte levels by taking multiple sequential measurements in various points in space and thus minimizing the impact of background. This rapid reversibility is exploited by the pattern recognition software to identify targets in unknown backgrounds.

### 7. Pattern Recognition Software

The challenge detection in plant environments goes beyond identification of single odorants, which has heretofore been the limit of ambition in this arena. The device meets the challenge presented by odor detection in real world environments where target odorants of importance occur along with a diversity of other sources in the presence of strong, unknown and changing background odorant landscape. The inventive system processes a series of sensor array snapshots with algorithms, which emulate the biological neural circuitry which has evolved to receive and transform the stream of signals delivered from the array of olfactory receptors.

### 8. The Integration of Both Mobile and Distributed Fixed Platforms

Sensor data from the distributed network of fixed, hand held and mobile sensors is wirelessly transmitted to a central controller where a dynamically updated map of target analyte concentration is maintained. Hand held mobile units may be augmented by a network of autonomous robotic mobile sensors, so that human beings are not required to physically explore and map dangerous odorant environments.

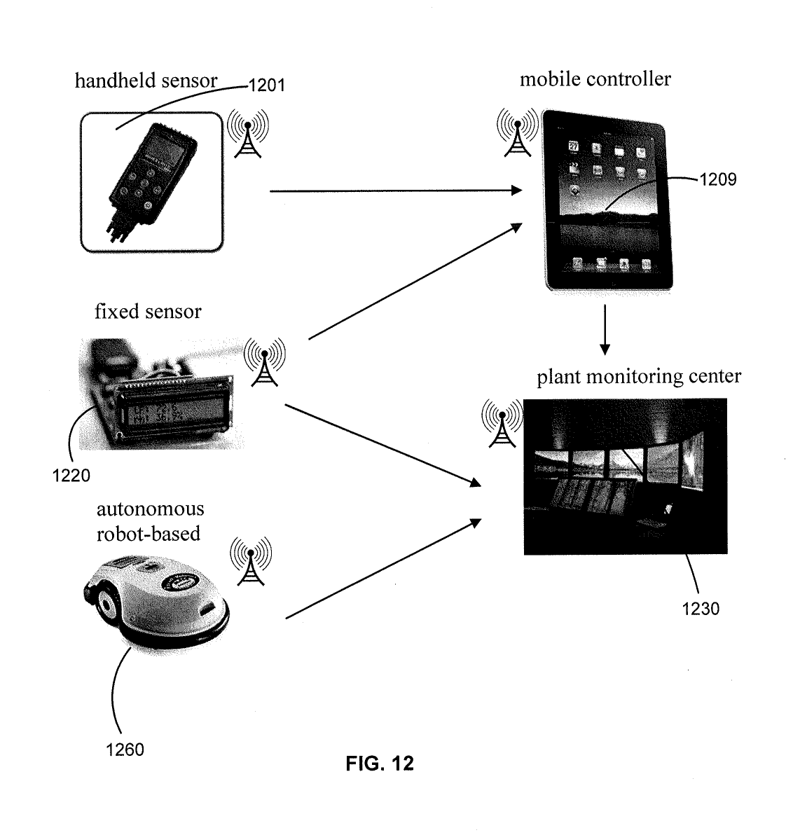

FIG. 12 show one embodiment of the distributed sensor system of the present invention. Hand held 1201 sensors can be used by individuals or clipped to their person as they do daily activities. A mobile controller 1209 is also deployed and is in wireless contact to the hand held sensors. The distributed system of the present invention is wirelessly connected in a distributed network. In addition, fixed sensors 1220 can monitor areas with a higher likelihood or probability of a leak occurring, or where a leak could cause the most damage to life and property. For example, a fixed system may be deployed in a high traffic area. Robot based sensors 1260 can be used in confined areas or where a leak is suspected and the analyte is toxic or dangerous. The plant monitoring center 1230 is the network hub and coordinates and deploys system activities.

The distributed sensing system 1200 can be used in many different applications. For example, the sensor units can be distributed across an oil refinery, oil field, chemical facility, and can be configured for fugitive emission detection and identification in order to monitor the leakage of volatile gases, oil spills or hazardous gases into the atmosphere. A valve failure, for example, will be discovered immediately and a technician or robot dispatched before it can present a serious safety hazard, life, property or seriously impact the operation of the operation. In this embodiment, the technician can have a handheld sensing device 1201 as described herein. At the scene of the leak or spill, the technician can assess the amount and nature of the leak. The handheld device can communicate with the network via wireless mode and the plant monitoring center 1230 or nerve center can dispatch the needed resources to contain the situation.

The system can also be used to monitor emission levels from industrial plants, mining operations ambient air monitoring, worker protection, emissions control, product quality testing, petrochemical applications and enclosed spaces. The data gathered can then be used to track and predict the progression of a plume of escaped dangerous or poisonous gas. In cases of an industrial incident or leak, this information can be used to first warn populations for evacuations and later to estimate the impact of the incident on those environments. In many industrial facilities, such as a nuclear facility, the sensor system can be used to monitor the perimeter.

The generated dynamic map, overlaid over a plant floor plan, is designed to enable a human controller as well as intelligent software to pinpoint the source of a leak, identify the spread of the plume, all to guide real-time rapid response to contain the leak and if need-be to respond rapidly to protect workers downstream. The handheld and fixed units are capable of transmitting sensor readings as well as receiving instructions from the central server, so that the handheld and wall-mounted units can guide field personnel to take the actions determined to best-respond to the situation as it develops, in real time. A record of the real-time dynamic map of odorant concentration can be stored and studied off-line to document the origin of the initial leak, and to support analysis necessary to prevent recurrence.

### 9. Summary

In a preferred aspect, the distributed sensor system comprises hybrid sensors on a semiconductor chip based on an array of nanotube field effect transistors functionalized with single-strand DNA. The detection and monitoring system comprises a distributed array of hundreds of these high dimensional, reversible, low power sensors, linked with wireless communication and animated by an innovative neural processing system. The inventive devices allow applications that require detection and identification of odors, for safety (searching for chemical or biological weapons, drugs, explosives), the food industry (wine, coffee, milk, juice quality control, taste control, control of ageing process of cheese or whisky), medicine (breath or sweat based diagnostic of cancer), air quality control in buildings or in closed accommodations.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A hybrid sensor array, said hybrid sensor array comprising:
a first hybrid sensor, wherein said first hybrid sensor has a first nanotube field effect transistor (NT FET) and a first companion transistor, wherein said first companion transistor addresses said first NT FET by gating a flow of current thereto; and
a second hybrid sensor, wherein said second hybrid sensor has a second NT FET and a second companion transistor, wherein said second companion transistor addresses said second NT FET by gating the flow of current thereto.

2. The hybrid sensor array of claim 1, wherein the nanotube of said first and said second NT FET may be the same or different and is a member selected from the group consisting of a single conducting nanotube, a single semiconducting nanotube, a mat of semiconducting nanotubes, and a mat of a combination of an intercalated semiconducting NT and a metallic nanotube.

3. The hybrid sensor array of claim 2, wherein said first NT FET and said second NT FET are each a carbon nanotube (CNT) FET.

4. The hybrid sensor array of claim 3, wherein said first CNT FET and said second CNT FET may be the same or different and each is a member of the group consisting of a single wall carbon nanotube (SWCNT), a multiwall carbon nanotube (MWCNT), graphene and a mixture thereof.

5. The hybrid sensor array of claim 4, wherein said first and said second CNT FET are each functionalized with a biomolecule.

6. The hybrid sensor array of claim 5, wherein said functionalized biomolecule is covalently or noncovalently bound to said first and said second CNT FET.

7. The hybrid sensor array of claim 5, wherein said functionalized biomolecule interacts with an analyte.

8. The hybrid sensor array of claim 7, wherein said interaction affects the current flow of said CNT FET.

9. The hybrid sensor array of claim 1, wherein said first companion transistor and said second companion transistor are each a thin film transistor (TFT).

10. The hybrid sensor array of claim 5, wherein said hybrid sensor array consists of at least n hybrid sensors, wherein n is a number in the range of about 4 to about 10⁵.

11. The hybrid sensor array of claim 10, wherein n is between at least 8 hybrid sensors to at least 64 hybrid sensors.

12. The hybrid sensor array of claim 10, wherein n is at least 16 hybrid sensors.

13. The hybrid sensor array of claim 12, wherein said at least 16 hybrid sensor array is a first hybrid sensor array in a first well of a multiwell sensor module.

14. They hybrid sensor array of claim 13, wherein each CNT FET of said at least 16 hybrid sensor array of the first hybrid sensor array is functionalized the same.

15. The hybrid sensor array of claim 13, wherein said sensor module has a second at least 16 hybrid sensor array in a second well of said multiwell sensor module.

16. The hybrid sensor array of claim 15, wherein each of said hybrid sensors of said second hybrid sensor array has a different functionalization than said first at least 16 hybrid sensor array.

17. The hybrid sensor array of claim 15, wherein said multiwell sensor module has y multiwells.

18. The hybrid sensor array of claim 17, wherein y of said multiwell sensor module is 96 wells.

19.-31. (canceled)

32. A method for detecting an analyte, said method comprising:
contacting said analyte with a hybrid sensor array, said hybrid sensor array comprising:
a first hybrid sensor, wherein said first hybrid sensor has a first nanotube field effect transistor (NT FET) and a first companion transistor, wherein said first companion transistor addresses said first NT FET by gating a flow of current thereto;
a second hybrid sensor, wherein said second hybrid sensor has a second NT FET and a second companion transistor, wherein said second companion transistor addresses said second CNT FET by gating the flow of current thereto;
affecting the current flow of said first and said second NT FET to generate a signal; and
detecting said signal thereby detecting said analyte.

33.-62. (canceled)

63. A distributed leak detection system of a defined geographical location to enable identification of a leak source, said leak detection system comprising:
a plurality of hybrid sensor arrays, wherein each of said hybrid sensor arrays comprises a first hybrid sensor, wherein said first hybrid sensor has a first nanotube field effect transistor (NT FET) and a first companion transistor, wherein said first companion transistor addresses said first NT FET by gating a flow of current thereto;
a second hybrid sensor, wherein said second hybrid sensor has a second NT FET and a second companion transistor, wherein said second companion transistor addresses said second NT FET by gating the flow of current thereto; and
the plurality of hybrid sensor arrays comprising at least one members selected from the group consisting of a stationary, a hand held, a mobile and a portable hybrid sensor array, wherein the plurality of hybrid sensors are recognizable on a map of said defined geographical location, wherein a leak affects the current flow of one of the plurality of hybrid sensors to generate a signal thereby detecting the leak.

64.-68. (canceled)
